(19) United States
(12) Patent Application Publication
(10) Pub. No.: US 2026/0358211 A1
(43) Pub. Date: Nov. 12, 2026

(54) AUTOMATED MATERIAL TESTING SYSTEMS AND ASSOCIATED METHODS

(71) Applicant: The Texas A&M University System, College Station, TX (US)

(72) Inventors: Jorge Roa, College Station, TX (US); Fujie Zhou, College Station, TX (US)

(73) Assignee: The Texas A&M University System, College Station, TX (US)

(21) Appl. No.: 19/126,454

(22) PCT Filed: Nov. 1, 2023
(86) PCT No.: PCT/US2023/078385

§ 371 Date: May 1, 2025

Related U.S. Application Data
(60) Provisional application No. 63/421,360, filed on Nov. 1, 2022.

(51) Int. Cl. G01N 3/04; G01N 3/08

(52) U.S. Cl. CPC G01N 3/04; G01N 3/08; G01N 2203/0062

(57) ABSTRACT

An automated material testing system includes a robotic arm including a base and a gripper pivotably coupled to the base, wherein the gripper has a defined field of motion, a void testing unit positioned at least partially within the field of motion of the gripper of the robotic arm, the void testing unit including a scale for weighing a material sample in both a dry state and a saturated surface dry (SSD) state, a material properties testing unit positioned at least partially within the field of motion of the gripper of the robotic arm, the materials property testing unit including sample mount for supporting a material sample during the performance of a material properties test on the material sample, and a controller including a processor and storage for storing instructions executable by processor to operate the robotic arm, the void testing unit, and the material properties testing unit.

### CROSS-REFERENCE TO RELATED APPLICATIONS

This application claims benefit of U.S. provisional patent application Ser. No. 63/421,360 filed Nov. 1, 2022, and entitled “Automated Material testing systems and Associated Methods,” which is hereby incorporated herein by reference in its entirety for all purposes.

### STATEMENT REGARDING FEDERALLY SPONSORED RESEARCH OR DEVELOPMENT

Not applicable.

### BACKGROUND

Material testing systems are utilized in diverse industries for determining the mechanical and physical properties of a wide range of different materials. These materials encompass but are not limited to naturally occurring materials and manmade materials like metals, polymers, composites, ceramics, and more. Material testing systems may determine the physical properties of different materials through the performance of one or more destructive and/or nondestructive tests of samples of the given material. In addition, material testing systems play a pivotal role in quality assurance, research, and development across industries such as manufacturing, construction, aerospace, and automotive engineering.

### BRIEF SUMMARY OF THE DISCLOSURE

An embodiment of an automated material testing system comprises a robotic arm comprising a base and a gripper pivotably coupled to the base, wherein the gripper has a defined field of motion, a void testing unit positioned at least partially within the field of motion of the gripper of the robotic arm, the void testing unit comprising a scale for weighing a material sample in both a dry state and a saturated surface dry (SSD) state, a material properties testing unit positioned at least partially within the field of motion of the gripper of the robotic arm, the materials property testing unit comprising sample mount for supporting a material sample during the performance of a material properties test on the material sample, and a controller comprising a processor and storage for storing instructions executable by processor to operate the robotic arm, the void testing unit, and the material properties testing unit. In some embodiments, the material properties testing unit comprises a tensile testing unit comprising at least one of a cracking mount and a rutting mount for performing at least one of an indirect tensile (IDT) strength test, an indirect tensile asphalt cracking test (IDEAL-CT), and an ideal rutting test (IDEAL-RT) on the material sample. In some embodiments, the tensile testing unit comprises a platform, a holder assembly supported on the platform and defining a receptacle configured to receive the material sample, the holder assembly comprising a cracking mount, a rutting mount, and an ejector unit, and a tensile load applicator coupled between the holder assembly and the platform and configured to move the holder assembly relative to the platform, wherein the cracking mount is moveable by an actuator of the holder assembly relative to the rutting mount. In certain embodiments, the cracking mount comprises an extended position corresponding to a first configuration of the tensile testing unit whereby the tensile testing unit is configured to perform either the IDT strength test or the IDEAL-CT, and a retracted position corresponding to a second configuration of the tensile testing unit whereby the tensile testing unit is configured to perform the IDEAL-RT. In certain embodiments, the system comprises a conditioning unit positioned at least partially within the field of motion of the gripper of the robotic arm, the conditioning unit configured to adjust the temperature of the material sample, without manual intervention, to meet a desired temperature. In some embodiments, the system comprises a sample drying unit positioned at least partially within the field of motion of the gripper of the robotic arm, the sample drying unit configured to dry the exterior of the material sample without manual intervention. In some embodiments, the sample drying unit comprises a towel belt and a drive unit configured to transport the towel belt along a closed loop path. In certain embodiments, the system comprises a radio-frequency identification (RFID) tracking system comprising a RFID reader configured to read one or more RFID tags attached to one or more of the material samples to identify the one or more material samples.

An embodiment of an automated testing system comprises a robotic arm comprising a base and a gripper pivotably coupled to the base, wherein the gripper has a defined field of motion, an automated loading system comprising a carousel within the field of motion of the robotic arm and having a plurality of receptacles for receiving one or more material samples, wherein the robotic arm is configured to retrieve the one or more material samples from the plurality of receptacles of the carousel, a material properties testing unit positioned at least partially within the field of motion of the gripper of the robotic arm, the materials property testing unit comprising sample mount for supporting a material sample during the performance of a material properties test on the material sample, and a controller comprising a processor and storage for storing instructions executable by processor to operate the robotic arm, the material properties testing unit, and the automated loading system. In certain embodiments, the loading system comprises a carousel upon which the plurality of receptacles are arranged, the carousel being rotatable about a rotational axis. In some embodiments, the loading system comprises a carousel motor in signal communication with the controller, and wherein the controller is configured to operate the carousel motor to rotate the carousel about the rotational axis and position a selected receptacle in a predefined loading position. In some embodiments, the carousel has a plurality of separate tiers spaced along the rotational axis, wherein at least some of the plurality of receptacles are arranged along each of the plurality of separate tiers. In certain embodiments, the system comprises a void testing unit positioned at least partially within the field of motion of the gripper of the robotic arm, the void testing unit comprising a scale for weighing a material sample in both a dry state and a saturated surface dry (SSD) state. In certain embodiments, the material properties testing unit comprises a tensile testing unit comprising at least one of a cracking mount and a rutting mount for performing at least one of an indirect tensile (IDT) strength test, an indirect tensile asphalt cracking test (IDEAL-CT), and an ideal rutting test (IDEAL-RT) on the material sample. In some embodiments, the system comprises a radio-frequency identification (RFID) tracking system comprising a RFID reader configured to read one or more RFID tags attached to one or more of the material samples to identify the one or more material samples.

An embodiment of an automated material testing system comprises a robotic arm comprising a base and a gripper pivotably coupled to the base, wherein the gripper has a defined field of motion, a radio-frequency identification (RFID) tracking system comprising a RFID reader configured to read one or more RFID tags attached to one or more corresponding material samples to identify the one or more material samples, a material properties testing unit positioned at least partially within the field of motion of the gripper of the robotic arm, the materials property testing unit comprising sample mount for supporting a material sample during the performance of a material properties test on the material sample, and a controller comprising a processor and storage for storing instructions executable by processor to operate the robotic arm, the material properties testing unit, and the RFID tracking system. In some embodiments, the controller is configured to correlate a test result of the material properties testing unit with a specific RFID tag of the one or more RFID tags to identify a specific material sample of the one or more material samples corresponding to the test result. In certain embodiments, the RFID reader is mounted at a predefined position for reading the one or more RFID tags. In certain embodiments, the one or more RFID tags stores identifying information of the one or more corresponding material samples. In some embodiments, the material properties testing unit comprises a tensile testing unit comprising at least one of a cracking mount and a rutting mount for performing at least one of an indirect tensile (IDT) strength test, an indirect tensile asphalt cracking test (IDEAL-CT), and an ideal rutting test (IDEAL-RT) on the material sample. In some embodiments, the system comprises a void testing unit positioned at least partially within the field of motion of the gripper of the robotic arm, the void testing unit comprising a scale for weighing a material sample in both a dry state and a saturated surface dry (SSD) state.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of exemplary embodiments of the disclosure, reference will now be made to the accompanying drawings in which:

FIG. 1 is a schematic view of an automated material testing system according to some embodiments;

FIG. 2 is a perspective view of an automated sample loading unit according to some embodiments;

FIG. 3 is a perspective view of an automated sample tracking unit according to some embodiments;

FIG. 4 is a perspective view of a void testing unit according to some embodiments;

FIG. 5 is a perspective view of a sample drying unit according to some embodiments;

FIGS. 6 and 7 are perspective views of another sample drying unit according to some embodiments;

FIG. 8 is a side cross-sectional view of the sample drying unit of FIGS. 6 and 7;

FIG. 9 is a perspective view of a sample conditioning unit according to some embodiments;

FIGS. 10 and 11 are perspective views of a tensile testing unit according to some embodiments;

FIG. 12 is a side view of the tensile testing unit of FIGS. 10 and 11;

FIG. 13 is a perspective view of the tensile testing unit of FIGS. 10 and 11 in a second configuration;

FIGS. 14 and 15 are top views of a docking system according to some embodiments;

FIG. 16 is a perspective view of a secondary connection of the docking system of FIGS. 14 and 15 according to some embodiments; and

FIG. 17 is a block diagram of a computer system according to some embodiments.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis. As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

As described above, material testing systems leverage nondestructive and/or destructive testing to determine mechanical and physical properties of a wide range of different materials. Material testing systems have undergone significant developments over the years, progressively advancing from rudimentary manual techniques to more sophisticated and automated approaches. The following is a survey of conventional material testing systems and their associated limitations, with a specific emphasis on the challenges posed by a lack of full automation.

In the early stages of material testing, manual methods prevailed, requiring operators to perform tests manually with basic instruments and record data through human observation. These methods were prone to inaccuracies, labor-intensive, and often lacked repeatability due to human factors. The need for increased precision and reliability spurred the emergence of semi-automated material testing systems. These systems included mechanical loading devices and data acquisition instruments. Nevertheless, these systems necessitated significant human intervention for sample preparation, loading, processing, and data analysis. Further, recent innovations have seen the integration of advanced technology into material testing systems. Modern systems now incorporate at least some computer control, precision sensors, and automation. These automated systems are capable of conducting a wide array of tests, including tensile, compression, bending, and fatigue tests on diverse materials.

To provide a particular example of a material testing system, some material testing systems are specifically adapted for the testing of asphalt (e.g., hot mix asphalt (HMA)) samples. Particularly, asphalt, including HMA, is often tested when designed (e.g., for a paved road or other application) to evaluate the performance characteristics of the asphalt. For example, asphalt when designed may be subject to a battery of separate tests including, for example, a void test to determine the air void content of the material sample, an indirect tensile (IDT) strength test, an indirect tensile asphalt cracking test (IDEAL-CT) to determine the asphalt's cracking tolerance, and/or an ideal rutting test (IDEAL-RT) to determine the asphalt's rutting tolerance where “rutting” refers to the formation of “ruts” or depressions in the asphalt in response to the application of pressure from road traffic and other sources. Conventionally, asphalt testing is typically performed at a testing or development facility and is generally a labor and time intensive process which may take a day or more to complete given that samples of the asphalt are manually prepared, manually transported between different testing machines, and manually loaded and removed from the testing machines of conventional material testing systems.

It may be understood the formulation of the asphalt may inadvertently vary at a production facility from the intended formulation engineered at the development facility. Thus, asphalt of a given formulation produced by a given production facility may not always have the same performance characteristics as intended during the initial testing and development of the given asphalt formulation. However, the time and expense associated with conventional material testing systems typically make it impractical to test asphalt at the production facility after the mix from which the asphalt is produced has been designed and tested at a separate development facility or laboratory.

Despite the progress in material testing systems (including material testing systems), numerous limitations endure, with particular focus on the challenges arising from the absence of complete automation. For example, many contemporary material testing systems still necessitate manual specimen loading. While some have automated load application, the alignment and positioning of specimens often depend on human operators, introducing inconsistency and limiting repeatability. This issue may be particularly prevalent in material testing systems which implement a battery of separate tests (e.g., the air void, cracking, rutting, and other tests performed by material testing systems) on one or more material samples, where the material samples must be manually loaded into each of the different tests performed by the given material testing system. In addition, although modern systems incorporate advanced sensors and data acquisition software, the interpretation and analysis of data may require human intervention, especially in the context of complex tests. This reliance on manual analysis can result in delays and subjectivity in reporting test results. Further, automated material testing systems may lack flexibility when faced with non-standard specimen shapes or evolving testing requirements. Such systems may encounter difficulties adapting to unique or changing industry standards.

Accordingly, embodiments disclosed herein include automated material testing systems including a controller for operating at least one of a robotic arm, a sample loading unit for loading one or more material samples, a tracking system for automatically tracking the different material samples used by the material testing system, and one or more separate testing units for performing one or more corresponding nondestructive and/or destructive tests on the material samples. In some embodiments, the one or more testing units of the automated material testing system includes a void testing unit, and a tensile testing unit for performing at least one of an indirect tensile (IDT) strength test, an indirect tensile asphalt cracking test (IDEAL-CT), and an ideal rutting test (IDEAL-RT) on a material sample. Embodiments of material testing systems may be configured to perform one or more different tests on material samples without the need for manual intervention.

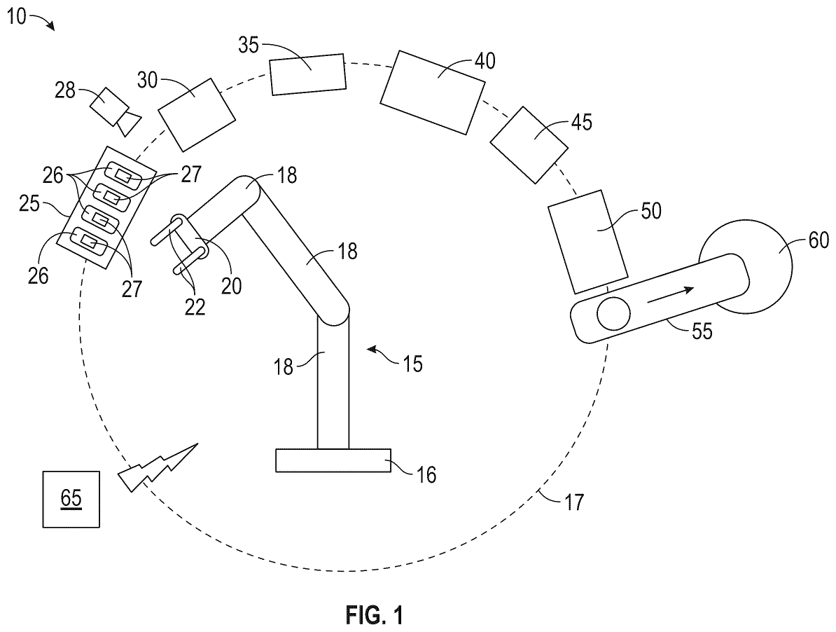

Referring to FIG. 1, an embodiment of an automated material testing system 10 is shown. In this exemplary embodiment, material testing system 10 is configured for testing asphalt (e.g., HMA) samples and thus is also referred to herein as asphalt testing system 10. However, it may be understood that material testing system 10 may be configured for testing various types of different materials (manmade and naturally occurring materials) beyond asphalt materials such as metals, polymers, composites, ceramics, concrete, aggregate, soil, and more.

Material testing system 10 includes a plurality of different automated units integrated seamlessly together such that one or more different tests may be performed on one or more different material samples without the need for human intervention (e.g., such as for retrieving a sample from a first test and loading the sample into a subsequent second test). Particularly, material testing system 10 generally includes a central robot or robotic arm 15, an automated sample loading unit 25, an automated sample tracking unit 28, a plurality of material properties testing units 30 and 50 and a control system or system controller 65. In this exemplary embodiment, material properties testing unit 30 comprises a void testing unit 30 while material properties testing unit 50 comprises a tensile testing unit 50; however, it may be understood that material properties testing units 30 and/or 50 may be configured to test other types of material properties other than for the presence of voids or tensile strength. In addition, in this exemplary embodiment, material testing system 10 includes a first drying unit 35, a sample conditioning unit 40, a second sample drying unit 45, a sample conveyor 55, and a spent sample receptacle 60. It may be understood that other embodiments of automated material testing systems consistent with the disclosure provided herein may not include each of the components shown in FIG. 1. It may similarly be understood that other embodiments of automated material testing systems consistent with this disclosure may include components in addition to those shown in FIG. 1.

In this exemplary embodiment, units 25, 30, 35, 40, 45, and 50 of material testing system 10 are arranged about the periphery of the robotic arm 15 of system 10. Robotic arm 15 generally includes a support base 16, a plurality of articulated arms or links 18 rotatably coupled to the support base 16, and an end-effector in the form of a gripper 20 pivotably coupled to the articulated links 18 and comprising a pair of opposed gripper pads 22. Support base 16 couples the robotic arm 15 to a support structure and may be stationary or mobile (e.g., transportable along rails) relative to the ground. Additionally, support base 16 includes a rotary actuator for rotating the articulated links 18 and gripper 20 relative to the ground about a vertically extending axis. Articulated links 18 are elongate in shape and pivotably coupled end-to-end via a plurality of rotary actuators located in the longitudinal ends of the articulated links 18. In this configuration, support base 16 and articulated links 18 may displace gripper 20 about a variety of separate axes to provide the gripper 20 with multiple degrees of freedom (DOF). For example, support base 16 and articulated links 18 may provide gripper 20 with six DOF; however, the kinds of motion provided to gripper 20 (e.g., the number of DOF provided to gripper 20) may vary in other embodiments depending on the configuration of support base 16 and the configuration and total number of articulated links 18 comprising the robotic arm 15.

The gripper 20 of robotic arm 15 is coupled to an end of one of the articulated links 18 and is configured to selectably grasp, manipulate, and transport material samples 26 (e.g., asphalt or HMA samples) which, in this exemplary embodiment, are disc-or puck-shaped (it being understood that the shape or geometry of the material samples may vary in other embodiments). Particularly, in this exemplary embodiment, gripper 20 comprises a pair of opposing gripper pads 22 configured to frictionally grasp an exterior of a given material sample 26. Gripper pads 22 may move along a common longitudinal axis towards and away from each other in order to selectably grasp a given material sample 26. For example, gripper pads 22 may move towards each other along the shared axis such that pads 22 press against opposing sides of a given material sample 26, securing the sample 26 to the gripper 20. Conversely, when secured to a given material sample 26, the gripper pads 22 may move away from each other along the common axis to release the given material sample 26 from the gripper 20. It may be understood that in other embodiments the configuration of gripper 20 (including number and the configuration of gripper pads 22) may vary in other embodiments.

As described above, units 25, 30, 35, 40, 45, and 50 of material testing system 10 are arranged about the periphery of robotic arm 15 whereby gripper 20 may access each of the units 25, 30, 35, 40, 45, and 50. Particularly, the gripper 20 of robotic arm 15 has a given field of motion indicated by the circle 17 in FIG. 1. In this exemplary embodiment, each of the units 25, 30, 35, 40, 45, and 50 comprising material testing system 10 are arranged to at least partially reside within the field of motion 17 of gripper 20 whereby gripper 20 may access each of the units 25, 30, 35, 40, 45, and 50 as will be discussed further herein. In this configuration, gripper 20 may, without manual intervention, transport a given material sample 26 between each of the units 25, 30, 35, 40, 45, and 50 comprising material testing system 10. Additionally, gripper 20 may, without manual intervention, load and unload a given material sample 26 from each of the units 25, 30, 35, 40, 45, and 50 comprising material testing system 10. To state in other words, robotic arm 15 may automatically, without manual intervention, perform one or more nondestructive and/or destructive tests on one or more material samples 26 using the units 25, 30, 35, 40, 45, and 50 comprising material testing system 10.

In this exemplary embodiment, the robotic arm 15 and one or more of the units 25, 30, 35, 40, 45, and 50 are controlled by the system controller 65 of material testing system 10. In some embodiments, system controller 65 comprises a computer system including a processor, memory, storage, one or more input/output (I/O) devices, and a communications device. The communication device of system controller 65 may be a wireless or wired communication device that may facilitate communication between the system controller 65 and one or more other components of material testing system 10 including, for example, robotic arm 15, sample loading unit 25, sample tracking unit 28, void testing unit 30, tensile testing unit 50, etc., as well as other computing devices The processor of system controller 65 may execute instructions stored on non-transitory memory thereof to control the operation of various components of material testing system 10. In addition, system controller 65 may process and store testing data or results obtained from the performance of one or more tests using the material testing system 10.

In this exemplary embodiment, the sample loading unit 25 of material testing system 10 receives one or more material samples 26 to be tested using the system 10. Sample loading unit 25 may include a support structure such as one or more stands or support frames for supporting the material samples 26 in any desired orientation. In some embodiments, material samples 26 may be manually delivered to the sample loading unit 25 by an operator of material testing system 10. Alternatively, material samples 26 may be delivered to the sample loading unit 25 without manual intervention using a conveyor for conveying or otherwise transporting the material samples 26 to the sample loading unit 25. Upon receiving one or more material samples 26, sample loading unit 25 is configured to sequentially position the received material samples 26 in a predefined loading position relative to the location of robotic arm 15 to permit the robotic arm 15 to unload, from the sample loading unit 25, a desired material sample 26 for loading into the void testing unit 30 of material testing system 10.

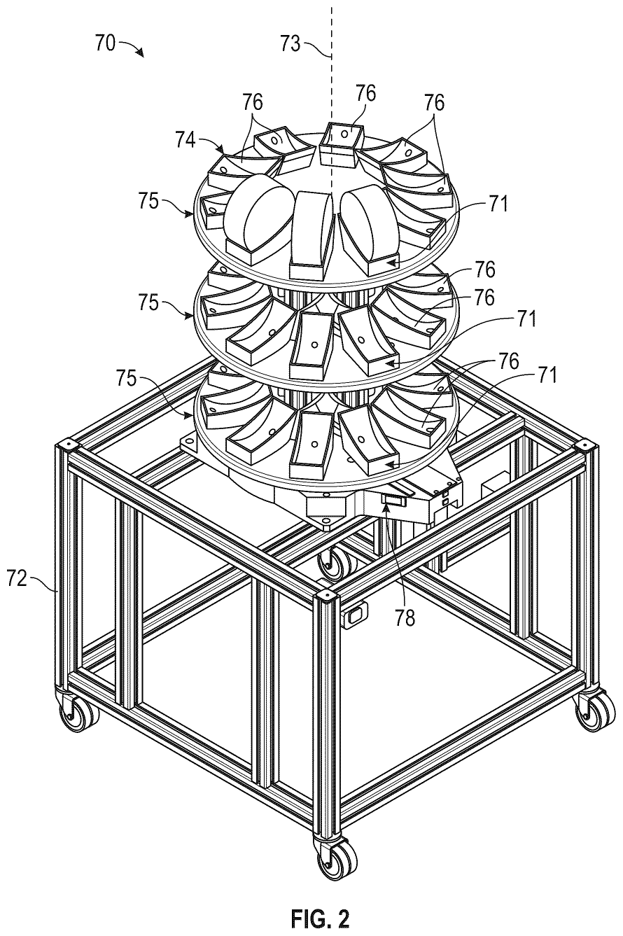

As an example, and referring now to FIG. 2, an embodiment of an automated sample loading unit 70 is shown. In some embodiments, the sample loading unit 25 of the material testing system 10 shown in FIG. 1 comprises or is configured similarly as the sample loading unit 70. In this exemplary embodiment, sample loading unit 70 generally includes a moveable (e.g., via one or more lockable wheels) support structure or dolly 72 and a sample loading carousel 74 positioned on the dolly 72.

The carousel 74 of sample loading unit 70 defines a plurality of sample receptacles 76 (only some of which are labeled in FIG. 2) located in different tiers 75 of the carousel 74. Particularly, sample receptacles 76 are positioned on carousel tiers 75 whereby sample receptacles 76 are circumferentially spaced around a centrally extending carousel axis 73 of the carousel 74. In some embodiments, material samples 26 are manually loaded into the corresponding sample receptacles 76. In other embodiments, material samples 26 may be loaded into sample receptacles 76 in an automated or semi-automated fashion using a robotic arm or other mechanism for loading the material samples 26 into sample receptacles 76. While in this exemplary embodiment sample unloading unit 70 includes carousel 74 which circumferentially arranges material samples 26 about the carousel axis 73, in other embodiments, sample loading unit 70 may include other mechanisms for arranging and holding material samples 26 to be loaded via a robotic arm or other mechanism. For example, in other embodiments, sample loading unit 70 may include a conveyor, a motion table, or other mechanism for selectably transporting one or more material samples 26 into one or more corresponding predefined sample loading positions.

Each sample receptacle 76 is shaped and configured to hold or retain a single material sample 26. In this exemplary embodiment, material samples 26 are disc or puck-shaped and each sample receptacle 76 is defined an internal curved, concave surface that receives a corresponding material sample 26 such that the sample 26 is held in a vertical orientation in the sample receptacle 76. However, it may be understood that the size, shape, or configuration of material samples 26 may vary in other embodiments with the size, shape, or configuration of sample receptacles 76 similarly varying in other embodiments to accommodate the given size, shape, or configuration of the material sample 26 received by the sample receptacle 76.

The tiers 75 are spaced vertically along the axis 73 of carousel 74. The use of vertically spaced tiers 75 provides carousel 74 with a relatively large number of sample receptacles 76 for receiving corresponding material samples 26. The number of tiers 75 of carousel 74 may vary depending on the requirements of the given application. Thus, in some embodiments, carousel 74 may include only a single tier 75 while in other embodiments carousel 74 may include more than three separate tiers 75. Each tier 75 of carousel is rotatable (together or individually) about axis 73 of carousel 74. Particularly, in this exemplary embodiment, carousel 74 comprises a carousel motor 78 for selectably rotating tiers 75 of carousel 74 about the carousel axis 73. For example, carousel motor 78 may comprise an electric motor such as a stepper motor controllable by a system controller (e.g., system controller 65 shown in FIG. 1).

Particularly, in this exemplary embodiment, each tier 75 defines its own sample loading position 71 in physical space that is predefined and fixed relative to the location of a robotic arm (e.g., robotic arm 15 shown in FIG. 1) used to unload selected material samples 26 from carousel 74. Specifically, each sample loading position 71 is disposed at a predefined circumferential location about the carousel axis 73 The robotic arm used to unload material samples 26 from carousel 74 may be trained to direct an end-effector thereof (e.g., gripper pads 22 of robotic arm 15 shown in FIG. 1) to the loading position 71 of a selected tier 75 in order to successfully receive a desired material sample 26 from the selected tier 75. For instance, the robotic arm may grasp a material sample 26 from the sample loading position 71 of an upper tier 75 of carousel 74. Alternatively, the robotic arm may grasp a material sample 26 from the sample loading position 71 of a middle tier 75 or a lower tier 75 of carousel 74. In this manner, the robotic arm knows precisely where to position its end-effector in order to unload a given material sample 26, minimizing the potential of the robotic arm failing to successfully unload a given material sample 26.

In some embodiments, each sample receptacle 76 of each carousel tier 75 may receive a corresponding material sample 26 and the robotic arm may (as instructed by the system controller), begin with unloading the material samples 26 of the upper tier 75 of carousel 74. For example, the robotic arm may unload a first material sample 26 initially residing in the sample loading position 71 of the upper tier 75. Once the first material sample 26 has been unloaded, carousel motor 78 may rotate the upper tier 75 of carousel 74 to position a second material sample 26 (previously positioned circumferentially adjacent to the first material sample 26) in the sample loading position 71 previously occupied by the now unloaded first material sample 26. The second material sample 26 may then be unloaded by the robotic arm and this process may be repeated until each of the material samples 26 held by the upper tier 75 of carousel 74 has been unloaded by the robotic arm. Once each material sample 26 of the upper tier 75 of carousel 74 has been unloaded, the robotic arm may (based on instructions provided by the system controller) begin unloading material samples 26 from the middle tier 75 of carousel 74, followed by the unloading of material samples 26 from the lower tier 75 of carousel 74. However, the order of unloading from carousel 74 may vary depending on the requirements of the given application.

Referring again to FIG. 1, the material samples 26 loaded using sample loading unit 25 are tracked via the sample tracking unit 28 of material testing system 10. In this manner, material testing system 10 may automate the collection of data obtained from the performance of one or more tests on the material samples 26 by automatically identifying and tracking the material samples 26 as they make their way through the material testing system 10.

As shown in FIG. 1, each material sample 26 includes an identifier or identification tag 27 affixed thereto which individually identifies the different material samples 26 handled by material testing system 10. As a selected material sample 26 is loaded from sample loading unit 25 by robotic arm 15, the sample tracking unit 28 scans the identification tag 27 of the loaded material sample 26 whereby the system controller 65 identifies the loaded material sample 26 such that controller 65 may automatically match data collected from the testing units 30 and 50 with the correct material sample 26 using the identification tag 27, minimizing the possibility of testing data being mistakenly associated (e.g., through human error) with an incorrect material sample 26.

In this exemplary embodiment, the sample tracking unit 28 includes a wireless sensor 29 for scanning the identification tags 27 of the material samples 26. In some embodiments, wireless sensor 29 comprises a radio frequency identification (RFID) scanner or reader and the identification tags 27 comprise RFID tags (e.g., passive RFID tags) which may be read wirelessly by the corresponding RFID reader 29. In other embodiments, wireless sensor 29 comprise an optical sensor or camera and the identification tags 27 of material samples 26 may comprise optically readable information (e.g., a barcode, a quick-response (QR) code) that may be read by the optical sensor. In still other embodiments, wireless sensor 29 may comprise other types of wireless sensors capable of wirelessly reading identification tags 27 such that the corresponding material samples 26 may be automatically read and tracked by sample tracking unit 28 without needing to interrupt or delay the operation of material testing system 10.

In addition, while in this exemplary embodiment the sample tracking unit 28 is positioned between the sample loading unit 25 and void testing unit 30, in other embodiments, the location of sample tracking unit 28 (e.g., relative to the other components or units of material testing system 10) may vary from that shown in FIG. 1. For instance, in other embodiments, sample tracking unit 28 may scan the identification tags 27 as or prior to the loading of material samples 26 into the sample loading unit 25.

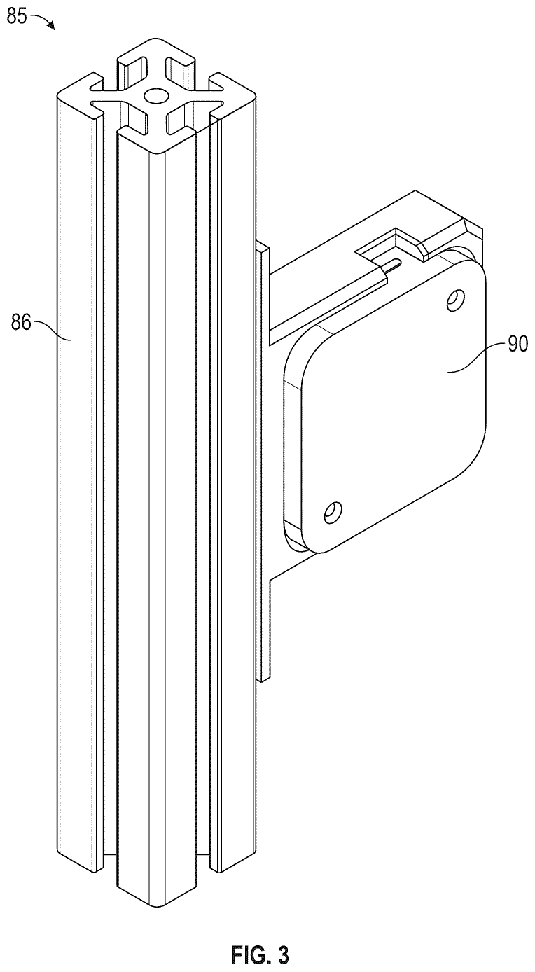

Referring now to FIG. 3, an embodiment of an automated sample tracking unit 85 is shown. In some embodiments, the sample tracking unit 28 of the material testing system 10 shown in FIG. 1 comprises or is configured similarly as the sample tracking unit 85. In this exemplary embodiment, sample tracking unit 85 generally includes a support structure or mount 86 and a wireless sensor 90 coupled to and supported by the mount 86. Mount 86 may be used to precisely position and mount the wireless sensor 90 of sample tracking unit 85 as desired by an operator of a material testing system incorporating sample tracking unit 85. For example, mount 86 may be coupled to a testing unit (e.g., void testing unit 30 shown in FIG. 1) in a location and orientation whereby wireless sensor 90 may seamlessly scan the identification tags 27 of material samples 26 as they are loaded (e.g., by a robotic arm) into the respective testing unit.

The wireless sensor 90 of sample tracking unit 85 is configured to wirelessly read the identification tags 27 placed on the material samples 26. Particularly, in this exemplary embodiment, wireless sensor 90 comprises a RFID reader and thus may also be referred to herein as RFID reader 90. RFID reader 90 is configured to emit (e.g., in response to instruction by a system controller) a wireless read signal that may be detected by the identification tag 27 of a selected material sample 26, causing the identification tag 27 to transmit a wireless response signal that is in-turn received by the RFID reader 90. The response signal may identify the material sample 26 corresponding to the given identification tag 27 and thus may allow the sample tracking unit 85 to positively identify different material samples 26 scanned by the sample tracking unit 85. In some embodiments, the response signal transmitted by a given identification tag 27 may include additional information characterizing the material sample 26 to which the identification tag 27 corresponds.

Referring again to FIG. 1, the void testing unit 30 of material testing system 10 is located adjacent sample loading unit 25 in this exemplary embodiment and is generally configured for determining the air void content of one or more material samples 26. As described above, in this exemplary embodiment, robotic arm 15 is configured to transport a given material sample 26 from the sample loading unit 25 and load the transported sample 26 into the void testing unit 30 whereby a void test may be performed by the void testing unit 30 on the transported material sample 26 to determine the air void content (e.g., as a percentage of the total volume of the material sample 26) of the sample 26.

In some embodiments, void testing unit 30 determines the air void content of a given material sample 26 by comparing the weight of the material sample 26 when dry and when in a saturated surface dry (SSD) state in which the exterior of the material sample 26 is dry (“surface dry”) while an interior of the material sample 26 is saturated with water (“saturated”). In certain embodiments, the void testing unit 30 is configured to, with assistance from the robotic arm 15 and the first sample drying unit 35 but without manual intervention, weigh a given material sample 26 when dry, internally saturate the material sample 26 with water, dry the surface or exterior of the material sample 26 while allowing the sample 26 to remain internally saturated with water whereby the material sample 26 is placed in the SSD state, and weigh the material sample 26 while in the SSD state. However, it may be understood that in other embodiments the process by which void testing unit 30 determines the air void content of a given material sample 26 may depart from the procedure outlined above.

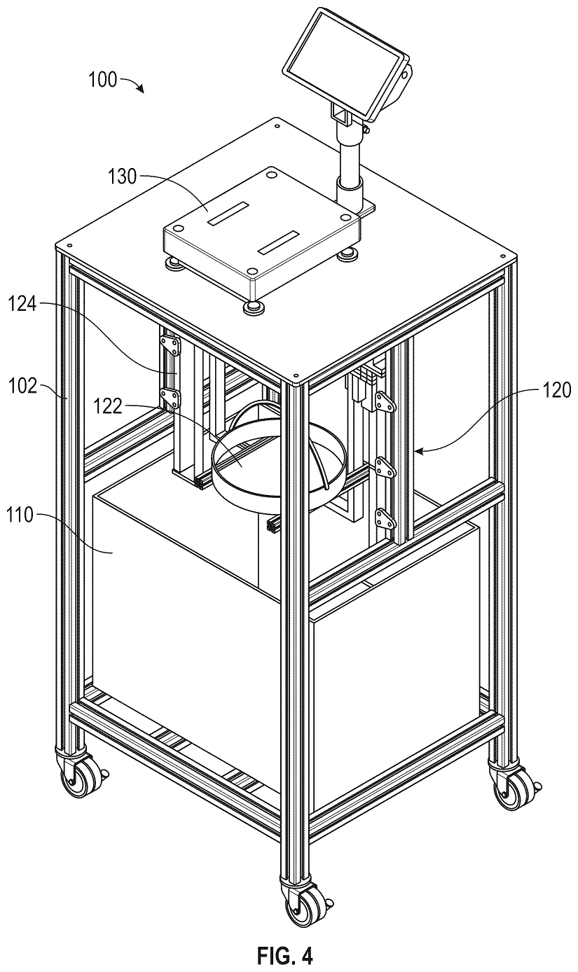

Referring now to FIG. 4, an embodiment of an automated void testing unit 100 is shown. In some embodiments, the void testing unit 30 of the material testing system 10 of FIG. 1 may be configured similarly as or comprise the void testing unit 100 shown in FIG. 4. In this exemplary embodiment, void testing unit 100 generally includes a support chassis or frame 102, a water tank 110 supported on the chassis 102, a dipping assembly or dipper 120, and a scale 130 for weighing material samples (e.g., material samples 26 shown in FIG. 1).

The water tank 110 of void testing unit 100 is supported on the chassis 102 and is at least partially fluid with a fluid (water) of a volume sufficient to completely submerge a given material sample when the material sample is dipped into the water tank 110 by the dipper 120. Additionally, dipper 120 is configured to selectably dip one or more material samples into the water tank 110 for a specified time period typically sufficient to entirely saturate the submerged material sample. In this exemplary embodiment, dipper 120 generally includes a tray 122 and an actuator assembly 124 coupled between the tray 122 and the chassis 102. Tray 122 is configured to receive one or more material samples and may be slotted, perforated, or otherwise may comprise openings for allowing fluid to drain from the tray 122 after being submerged in the water tank 110. Actuator assembly 124 may include one or more linear actuators for transporting the tray 122 vertically upwards and downwards relative to the water tank 110. Actuator assembly 124 may be controlled by a controller (e.g., system controller 65) as part of an automated routine executed by the controller.

The scale 130 of void testing unit 100 is a digital scale that may be in signal communication with a controller (e.g., system controller 65) for receiving, storing, and/or processing the measurements (weighing of material samples in a dry state, saturated state, and a SSD state) performed by scale 130. It may be understood that a robotic arm (e.g., robotic arm 15 shown in FIG. 1) may be used, without manual intervention, to load or remove a given material sample onto or from the tray 122 of dipper 120 and/or the scale 130.

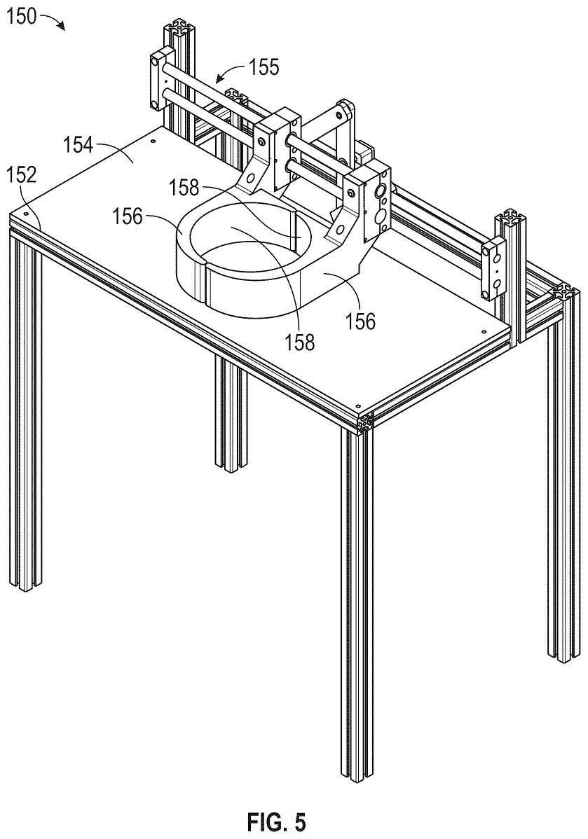

Referring now to FIGS. 4 and 5, an embodiment of a sample drying unit 150 is shown in FIG. 5 which may be used collectively with the void testing unit 100 shown in FIG. 4. In some embodiments, one of and/or both of the sample drying units 35 and 46 of the material testing system 10 of FIG. 1 may be configured similarly as or comprise the sample drying unit 150 shown in FIG. 5. In this exemplary embodiment, sample drying unit 150 includes a platform or table 152, an actuator assembly 155 supported on the platform 152, and a pair of drying pads 156 each coupled to the actuator assembly 155 and supported on the platform 152.

The platform 152 of sample drying unit 150 includes an upper sample support surface 154 upon which a material sample (e.g., material samples 26 shown in FIG. 1) may be positioned using a robotic arm (e.g., robotic arm 15 shown in FIG. 1). The sample support surface 154 may be lined or otherwise comprise an absorbent material (e.g., an absorbent fabric) configured to absorb moisture from the exteriors of material samples positioned on the support surface 154. For example, material samples such as the material samples 26 described above may be puck-shaped having an exterior defined by a pair of opposing planar surfaces and a curved surface extending about the periphery of the material sample. Sample support surface 154 may be used to dry each opposing planar surface of such a material sample by sequentially positioning the surfaces (e.g., via flipping the material sample over using a robotic arm) on the sample support surface 154 for a time period sufficient to dry the opposing planar surfaces.

The drying pads 156 of sample drying unit 150 are configured to dry the portions of the material sample that are not dried by sample support surface 154, such as the curved exterior surface of the puck-shaped material samples 26. Particularly, actuator assembly 155 comprises one or more linear actuators for transporting the drying pads 156 along a common horizontally extending axis between an open configuration and a closed configuration (shown in FIG. 5). It may be understood that actuator assembly 155 may be controlled by a separate controller (e.g., system controller 65) as part of an automated routine executed by the controller.

In the open configuration an opening is formed between the pair of drying pads 156 in which a material sample may be deposited, such as by a robotic arm transporting the material sample from the void testing unit 100 after the sample has been submerged in the water tank 110 thereof. With a material sample positioned on the sample support surface 154 between the pair of drying pads 156, the actuator assembly 155 may shift drying pads 156 from the open configuration to the closed configuration whereby curved inner surfaces 158 of the drying pads 156 press against the curved exterior of the material sample. As with the sample support surface 154, the inner surfaces 158 of drying pads 156 may be lined or otherwise comprise an absorbent material (e.g., an absorbent fabric) configured to absorb moisture from the exteriors of material samples contacted by the drying pads 156. After a predetermined drying time period, the drying pads 156 may be shifted by actuator assembly 155 from the closed configuration to the open configuration so that the dried material sample may be retrieved by a robotic arm. Although drying pads 156 comprise curved inner surfaces 158 in this exemplary embodiment, it may be understood that in other embodiments the shape of drying pads 156 may vary in accordance with the shape or configuration of the given material samples dried by the sample drying unit 150.

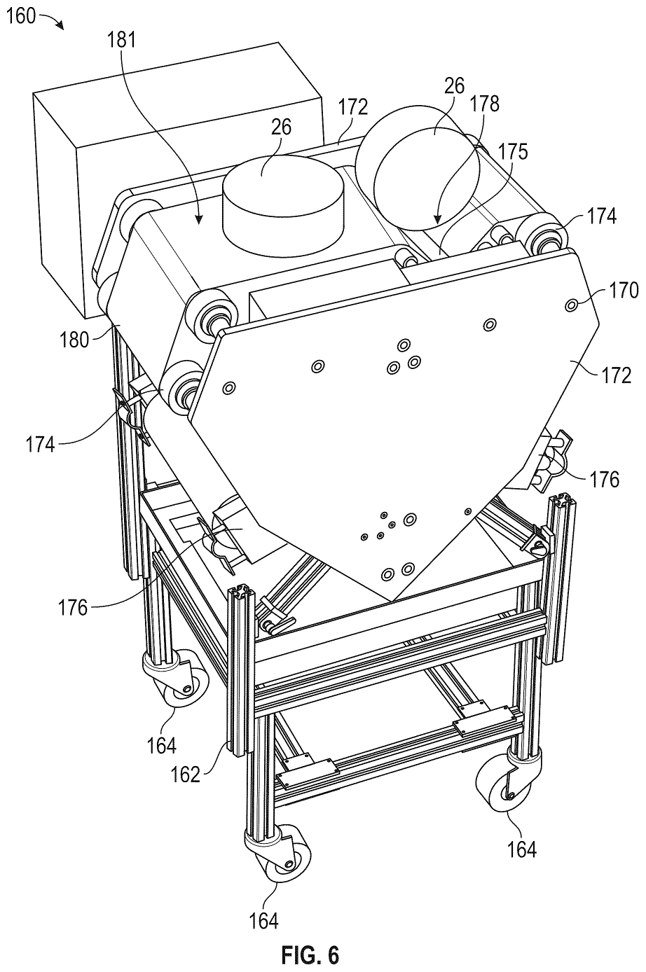

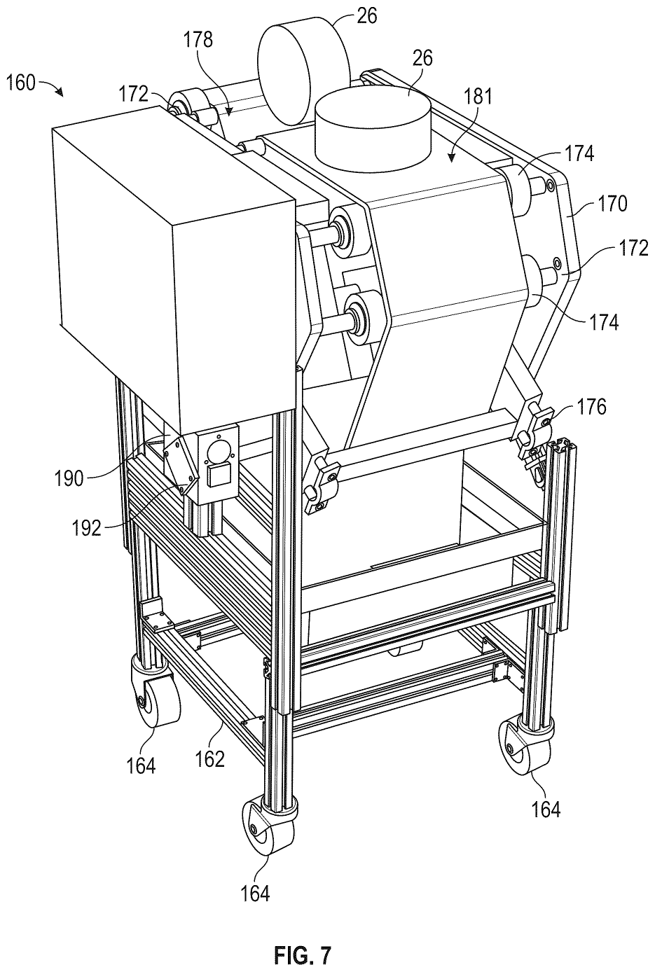

Referring now to FIGS. 6-8, another embodiment of a sample drying unit 160 is shown in FIG. 5 which may be used collectively with the void testing unit 100 shown in FIG. 4. In some embodiments, one of and/or both of the sample drying units 35 and 45 of the material testing system 10 of FIG. 1 may be configured similarly as or comprise the sample drying unit 160 shown in FIGS. 6-8. In this exemplary embodiment, sample drying unit 160 includes a platform or dolly 162, a dryer housing 170 positioned on the platform 162, an endless or continuous towel belt 180 supported by the dryer housing 170, and a drive unit 190 for transporting and wringing the towel belt 180. The platform 162 physically supports the remaining components of the drying unit 160 and, in this exemplary embodiment, is portable via a plurality of wheels 164 located at a bottom of the platform 162.

The dryer housing 170 is positioned atop the platform 162 and generally includes a pair of generally planar and opposing support panels 172, a plurality of idle rollers 174, a plurality of receptacle rollers 175, a pair of tensioner 176, and a pair of drive roller assemblies 194. The pair of support panels 172 physically support the rollers 174, 175 tensioners 176, and drive roller assemblies 194 where each of the components 174-176, and 194 extend longitudinally between the pair of support panels 172. In addition, components 174-176 and 194 are permitted to rotate about their respective axes of rotation relative to the pair of support panels 172 such that components 174-176 and 194 may be said to be rollably coupled to the pair of support panels 172.

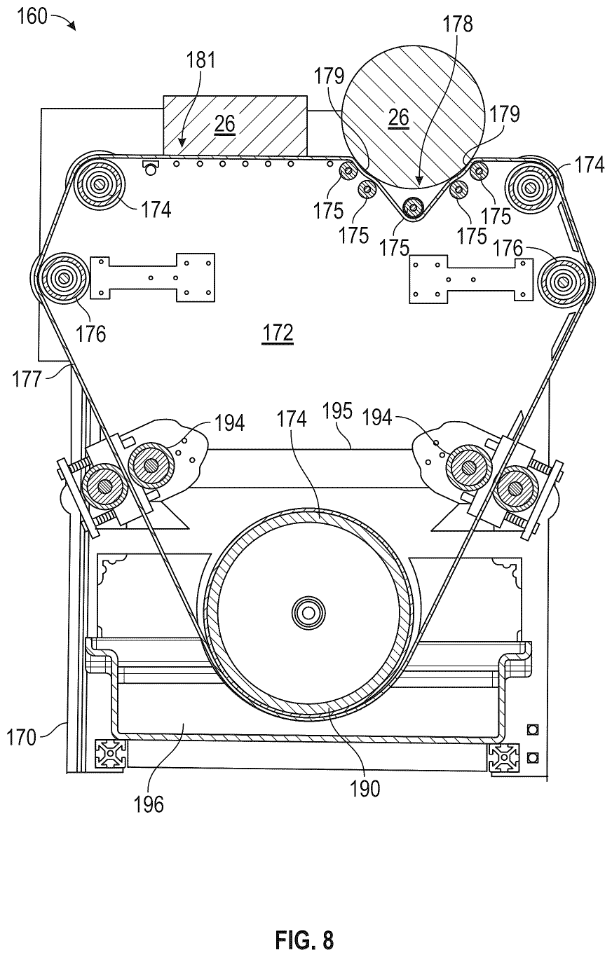

The rollers 174, 175 and tensioners 176 assist in defining a closed loop path (indicated by arrow 177 in FIG. 8) along which the towel belt 180 extends. Particularly, in this exemplary embodiment, the receptacle rollers 175 which comprise idler rollers permitted to freely spin about their respective rotational axes, define a sample receptacle 178 along an upper end of the path 177. The sample receptacle 178 is configured to partially receive a material sample 26 in a vertical orientation whereby a section of the annular outer surface or periphery of the material sample 26 rests against the portion of towel belt 180 forming the sample receptacle 178. In this exemplary embodiment, sample receptacle 178 has a V-shaped side profile (shown in FIG. 8) to partially receive the vertically oriented material sample 26. In this manner, a pair of opposing inclined (e.g., relative to the direction of gravity) surfaces 179 of the sample receptacle 178 support the vertically oriented material sample 26 such that the sample 26 may be maintained in the vertical orientation without falling during the operation of sample drying unit 160. However, it may be understood that the geometry of sample receptacle 181 may vary in other embodiments to accommodate differently shaped material samples.

As with receptacle rollers 175, the idler rollers 174 and tensioners 176 are also permitted to freely spin about their respective rotational axes. Idler rollers 174 physically support the towel belt 180 along the closed loop path 177 and tensioners xxx are configured to tension the towel belt 180 and thereby reduce slack in the towel belt 180 during operation of the sample drying unit 160. Further, in addition to sample receptacle 178, the closed loop path 177 defines a generally flat or planar sample platform 181 along the upper end of closed loop path 177 and adjacent or proximal to the sample receptacle 178. Sample platform 181 is configured to receive a material sample 26 in a horizontal orientation whereby the material sample 26 in a horizontal orientation may be positioned on a section of the towel belt 180 forming the sample platform 181 such that one of the planar faces of the material sample 26 contacts the towel belt 180.

The towel belt 180 of sample drying unit 160 forms a closed loop along closed loop path 177 and comprises an absorbent material (e.g., an absorbent fabric or other material) for absorbing moisture located along the external surface of different material samples 26 via physically contacting the material sample 26 with the towel belt 180.

The drive unit 190 of sample drying unit 160 is configured to drive the towel belt 180 continuously along the closed loop path 177 whereby the towel belt 180 may be used to dry one or more different material samples 26. In this exemplary embodiment, drive unit 190 generally includes a drive motor 192 (e.g., an electric motor) and a pair of drive roller assemblies 194 coupled to an output shaft of the drive motor 192. Particularly, each drive roller assembly 194 comprises a pair of separate, rotatable drive rollers with the towel belt 180 passing between the pair of drive rollers. The drive roller assemblies 194 are connected together by a drive belt 195 coupled to one drive roller of each drive roller assembly 194. In addition, in this exemplary embodiment, drive roller assemblies 194 compress (e.g., via a biasing member of each drive roller assembly 194) against the towel belt 180 passing between the pair of drive rollers of the given drive roller assembly 194. The compression provided by drive roller assemblies 194 wring moisture from the towel belt 180 as the towel belt 180 travels along closed loop path 177 whereby the extracted moisture drops and collects within a fluid reservoir 196 of the sample drying unit 160 located at a lower end or bottom of the frame 170.

Drive motor 192 is configured to continuously rotate (e.g., in response to receiving a control signal from a system controller such as the system controller 65 shown in FIG. 1) one of the drive roller assemblies 194 about its rotational axis where motion of the driven drive roller assembly 194 is transferred to the other drive roller assembly 194 by the drive belt 195 connected therebetween. Thus, drive motor 192 may be in signal communication with a system controller for controlling the operation of drive motor 192. In turn, the drive rollers of both drive roller assemblies 194 are coupled (e.g., via friction or other means) to the towel belt 180 whereby rotation of drive rollers of drive roller assemblies 194 about their rotational axes force towel belt 180 to travel along the closed loop path 177. Thus, activation of the drive motor 192 results, via the operation of drive roller assemblies 194, in the movement of towel belt 180 along closed loop path 177.

During operation of sample drying unit 160, unit 160 may be used to dry each of the external surfaces of one or more material samples 26, including the annular periphery and planar faces of each material sample 26. Particularly, in an embodiment, a robotic arm (e.g., robotic arm 15 shown in FIG. 1) may grasp a material sample 26 and position the material sample 26 in a vertical orientation in the sample receptacle 178 of sample drying unit 160. Once the robotic arm releases the vertically oriented material sample 26, the drive unit 190 may be activated (e.g., via a system controller such as system controller 65 shown in FIG. 1) to drive the towel belt 180 along the closed loop path 177. In turn, motion of the towel belt 180 along closed loop path 177 result in the stationary rotation (e.g., rotation about a horizontal axis extending centrally through the sample 26) of the vertically oriented material sample 26 received in the sample receptacle 178 whereby the entire circumferential periphery of the material sample 26 contacts and is dried by the towel belt 180.

In addition, in an embodiment, activation of the drive unit 190 may be ceased and the robotic arm may pick up the material sample 26 (now having its circumferential outer periphery dried by the sample drying unit 160) and position the material sample 26 now in a horizontal orientation on the sample platform 181 of sample drying unit 160 to dry a first planar face of the material sample 26. After the first planar face of the material sample 26 has dried, the robotic arm may again pick up the material sample 26 as the drive unit 190 is again activated to briefly drive movement of the towel belt 180 along the closed loop path 177 such that an unused section of the belt 180 is now positioned along the sample platform 181. Subsequently, the robotic am may lower the material sample and position the second planar face of the material sample 26 into contact with towel belt 180 along the sample platform 181 whereby drying of the material sample 26 by the sample drying unit 160 may be successfully completed.

As described above, in addition to driving the motion of towel belt 180, drive assemblies 194 act as wringers to extract excess moisture from the towel belt 180 as the towel belt 180 travels along closed loop path 177. Particularly, as towel belt 180 travels along path 177, towel belt 180 travels through the fluid (e.g., water) collected in fluid reservoir 196 thereby saturating the towel belt 180 with fluid. This saturated section of towel belt 180 passes through one of the drive assemblies 194 prior to reaching the upper end of closed loop path 177 (irrespective of the direction of travel of towel belt 180 along path 177) whereby excess moisture is extracted from the towel belt 180. However, the amount of moisture extracted from towel belt 180 by drive roller assemblies 194 is controlled (e.g., via controlling the amount of compression applied by the drive rollers of assemblies 194 against the towel belt 180) such that a predefined moisture level is maintained in the towel belt 180 as it arrives at the upper end of closed loop path 177 where it may come into contact with a material sample 26. By maintaining a predefined moisture level in the towel belt 180 as it is contacted by the material samples 26, sample drying unit 160 thereby ensures that each material sample 26 is dried only to the extent that the sample 26 is placed in a SSD state. In other words, the wringing action provided by drive roller assemblies 194 ensures that each material sample 26 dried by sample drying unit 160 is not dried too little (resulting in excess moisture on the external surfaces of the sample 26) or too much (such that the sample 226 is no longer saturated), thereby enhancing the accuracy of the testing performed on the material samples 26 placed in the SSD state by the sample drying unit 160.

Further, at some point in the operational life of sample drying unit 160, it may be desired to replace the towel belt 180. In this exemplary embodiment, towel belt 180 may be quickly and conveniently removed from the sample drying unit 160 simply by releasing the tensioners 176 to induce sufficient slack in the towel belt 180 where it may be manually removed from the sample drying unit 160. A replacement towel belt 180 may then be quickly installed in the sample drying unit 160 with tension reapplied by tensioners 176 and drive roller assemblies 194. Referring again to FIG. 1, as described above, the robotic arm 15, void testing unit 30, first sample drying unit 35, and system controller 65 may be used for determining, without manual intervention, the air void content of a given material sample 26. Particularly, the system controller 65 may compare the dry and SSD weights of each measured material sample 26 and may save the determined air void content data in a storage device thereof.

Following the void testing performed at least partially by void testing unit 30, a given material sample 26 may be transported by the gripper 20 of robotic arm 15 from the void testing unit 30 to the conditioning unit 40 of material testing system 10. Conditioning unit 40 may condition one or more material samples 26 at a given time to prepare the material samples 26 for the testing performed by tensile testing unit 50. For example, conditioning unit 40 may adjust a temperature of a given material sample 26 by cooling or heating the material sample 26 such that the temperature of the sample 26 corresponds to a temperature prescribed by one of the tests performed by tensile testing unit 50. Additionally, in some embodiments, conditioning unit 40 may saturate a given material sample 26 with liquid (e.g., water) as the temperature of the material sample 26 is adjusted by the conditioning unit 40.

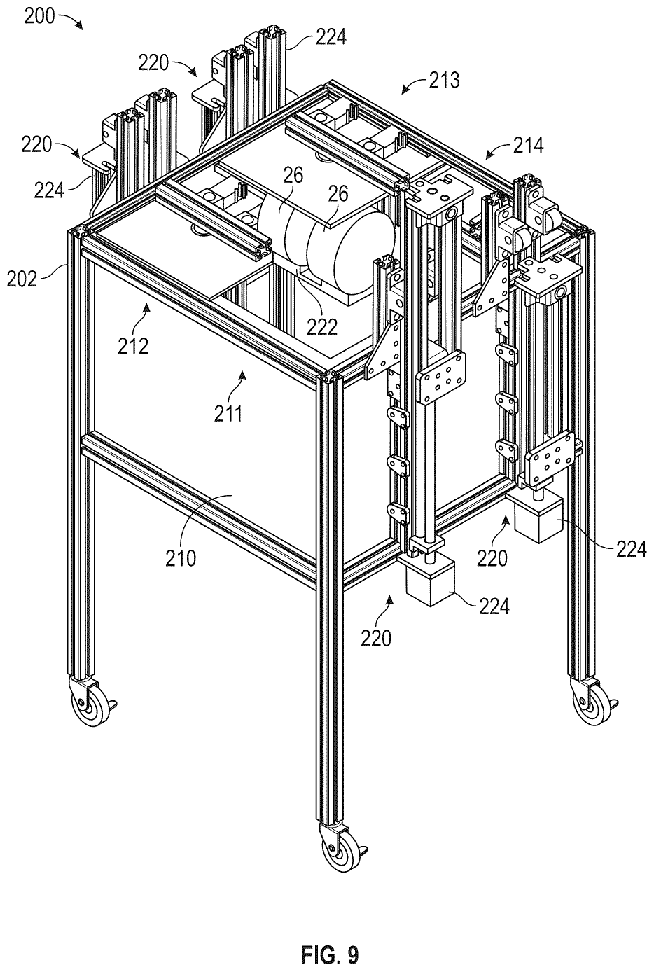

Referring now to FIG. 9, an embodiment of an automated conditioning unit 200 is shown. In some embodiments, the conditioning unit 40 of the material testing system 10 of FIG. 1 may be configured similarly as or comprise the conditioning unit 200 shown in FIG. 9. In this exemplary embodiment, conditioning unit 200 generally includes a support chassis or frame 202, a tank 210 supported by the chassis 202 and defining a plurality of separate chambers 211-214, and a plurality of dipping assemblies or dippers 220 associated with the chambers 211-214 of the conditioning unit 200. Each chamber 211-214 of tank 210 may at least be partially filled with water independently maintained at a desired temperature. For example, a first chamber 211 may be maintained at an elevated temperature (e.g., 120 °F) while a second chamber 212 may be maintained at room temperature (e.g., 77 °F) while a third chamber 213 may be maintained at a cold temperature (e.g., 50 °F) that is less than room temperature. Tank 210 may comprise one or more heating and/or cooling sources such as one or more heat pumps or the like for independently controlling the temperatures of chambers 211-214, where the one or more heating and/or cooling elements may be controlled by a controller (e.g., system controller 65 shown in FIG. 1).

Each, dipper 220 of condition unit 200 is associated with a different chamber 211-214 of tank 210 whereby a given dipper 220 configured to selectably dip one or more material samples into the chamber 211-214 associated with the dipper 220 for a specified time period typically sufficient to entirely saturate the submerged material sample. In this exemplary embodiment, each dipper 220 generally includes a tray 222 (only one of which is visible in FIG. 9) and an actuator assembly 224 coupled between the tray 122 and the chassis 102. Tray 222 is configured to receive one or more material samples in a vertical orientation in this exemplary embodiment (a pair of exemplary material samples 26 are shown in a vertical orientation in FIG. 9 in the interest of clarity). Additionally, each tray 222 may include a cover or enclosure intended to enclose the chamber 211-214 associated with the given dipper 220 when the tray 222 is submerged within the chamber 211-214. The enclosure of the tray 222 may assist in regulating the temperature of the chamber 211-214 associated with the given dipper 220. The actuator assembly 224 of each dipper 220 may include one or more linear actuators for transporting the tray 222 vertically upwards and downwards relative to the tank 210. Additionally, the actuator assembly 224 of each dipper 220 may be controlled by a controller (e.g., system controller 65) as part of an automated routine executed by the controller. Further, a robotic arm (e.g., robotic arm 15 shown in FIG. 1) may be used for loading material samples vertically into the tray 222 of a given dipper 220 and for removing the material samples from the tray 222.

Referring again to FIG. 1, in some embodiments, following conditioning of a material sample 26 in the sample conditioning unit 40, the exterior of the material sample 26 is dried in the sample drying unit 45 positioned adjacent the sample conditioning unit 40. As with the sample drying unit 35 described above, sample conditioning unit 45 may dry the exterior of a given material sample 26 while permitting the interior of the material sample 26 to remain saturated with fluid, thereby placing the material sample 26 into a SSD state. In certain embodiments, sample drying unit 45 dries the material sample 26 with the sample 26 in the vertical orientation. Sample drying unit 45 may be configured similarly as the sample drying unit 150 shown in FIG. 5 except that the drying pads of sample drying unit 45 may be rotated approximately ninety degrees to permit the sample drying unit 45 to dry a material sample 26 while in the vertical orientation with the curved surface of the material sample 26 positioned at the vertically lower and upper ends of the material sample 26. In addition to moveable drying pads, sample drying unit 45 may include vertically extending drying surfaces meant to dry the opposing planar surfaces of a material sample 26 while in the vertical orientation.

Robotic arm 15, having transferred a conditioned material sample 26 from the sample conditioning unit 40 to the sample drying unit 45, may, without manual intervention, retrieve the dried material sample 26 from the sample drying unit 45 and load the retried material sample 26 into the tensile testing unit 50 for tensile testing. In some embodiments, tensile testing unit 50 is configured to perform, without manual intervention, an IDT strength test, an IDEAL-CT test, and/or an IDEAL-RT test on the conditioned and dried material sample 26. The operation of tensile testing unit 50 may be controlled by controller 65 as part of an automated routine. Additionally, data collected by tensile testing unit 50 from the performance of an IDT strength test, an IDEAL-CT test, and/or an IDEAL-RT test may be collected and stored by the system controller 65.

In certain embodiments, tensile testing unit 50 is reconfigurable between a first configuration in which the tensile testing unit 50 is configured to perform an IDT strength test or an IDEAL-CT test on a material sample 26, and a second configuration in which the tensile testing unit 50 is configured to perform an IDEAL-RT test on a material sample 26, where system controller 65 of material testing system 10 may control which of the two configurations the tensile testing unit 50 occupies at a given time. In this manner, tensile testing unit 50 may be placed in the first configuration to perform an IDT strength test or an IDEAL-CT test on a first material sample 26, and subsequently shifted to the second configuration to perform an IDEAL-RT test on a second material sample 26.

It may be understood that the IDT strength test, IDEAL-CT test, and/or IDEAL-RT tests performable by tensile testing unit 50 are destructive tests which damage or destroy (e.g., crack or rut) the material samples 26 upon which the tests are performed. In this exemplary embodiment, conveyor 55 of material testing system 10 is provided directly adjacent to the tensile testing unit 50 for the purpose of automatically transporting “spent” material samples 26 (the remains of a material sample 26 following the performance of an IDT strength test, an IDEAL-CT, and/or an IDEAL-RT test) from the tensile testing unit 50 to the spent sample receptacle 60. It may be understood that in other embodiments the spent material samples 26 may be manually removed from tensile testing unit 50 or removed via an automated device which differs from the conveyor 55 shown in FIG. 1.

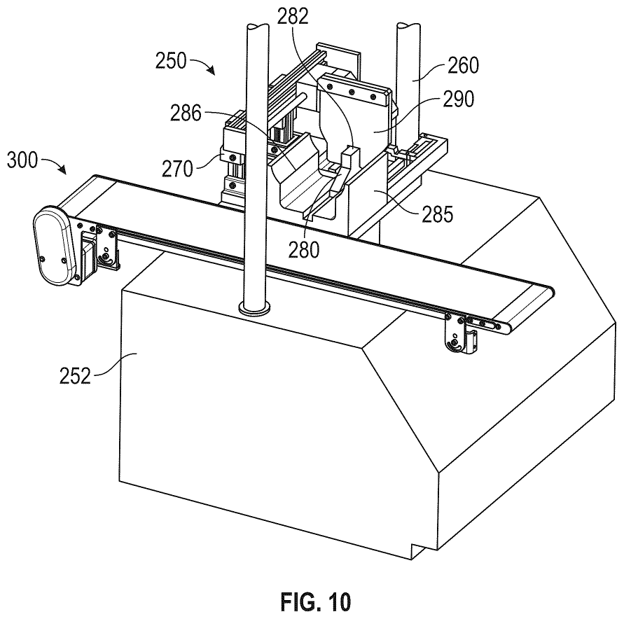

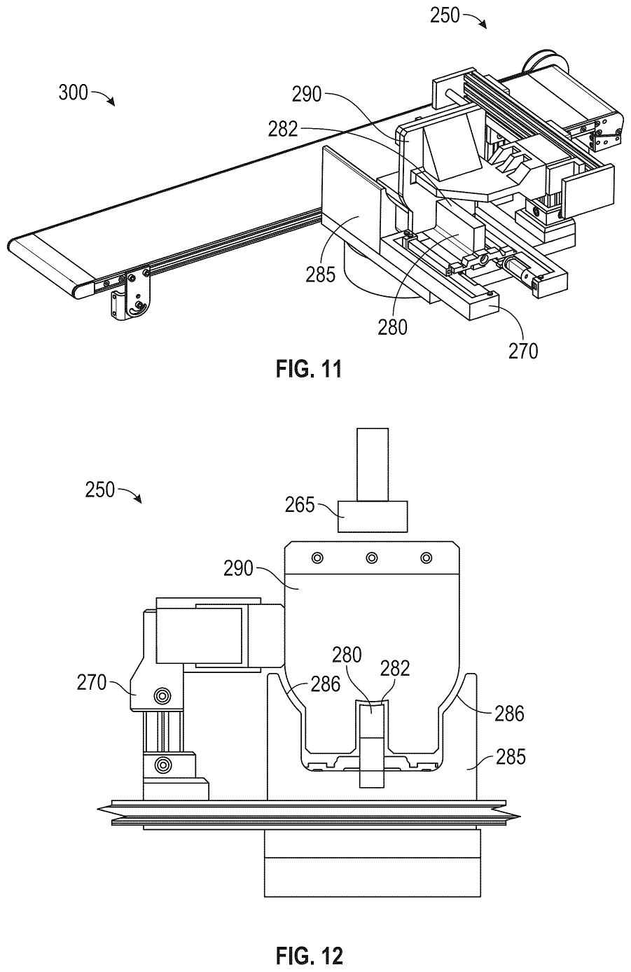

Referring now to FIGS. 10-13, an embodiment of an automated tensile testing unit 250 is shown for performing IDT strength, IDEAL-CT, and IDEAL-RT tests on material samples (e.g., the material samples 26 shown in FIG. 1). Additionally, an embodiment of a conveyor 300 is shown in FIGS. 10, 11, and 13; however, it may be understood that in some embodiments tensile testing unit 250 may not be paired with conveyor 300 as shown in FIGS. 10, 12, and 13. In this exemplary embodiment, tensile testing unit 250 generally includes a table or platform 252, one or more tensile load applicators 260, a sample holder assembly 270, a cracking mount 280, a rutting mount 285, and an ejector unit 290.

The tensile load applicators 260 are coupled between the platform 252 of tensile testing unit 250 and the holder assembly 270. Tensile load applicators 260 may comprise linear actuators such as lead screws and the like for transporting the holder assembly 270 vertically relative to the platform 252. Additionally, tensile testing unit 250 includes a mechanical stop 265 (shown in FIG. 12 but hidden from view in FIGS. 10, 11, and 13) also supported on the platform 252 but which does not move in response to actuation of the tensile load applicators 260. In this configuration, actuation of tensile load applicators 260 causes holder assembly 270 (and any material sample loaded therein) vertically towards or away from the fixed mechanical stop 265. In this manner, a tensile load may be applied to a material sample loaded into the holder assembly 270 in response to the actuation of tensile load applicators 260. It may be understood that the operation of load applicators 260 may be controlled by a controller (e.g., the system controller 65 shown in FIG. 1) as part of an automated routine.

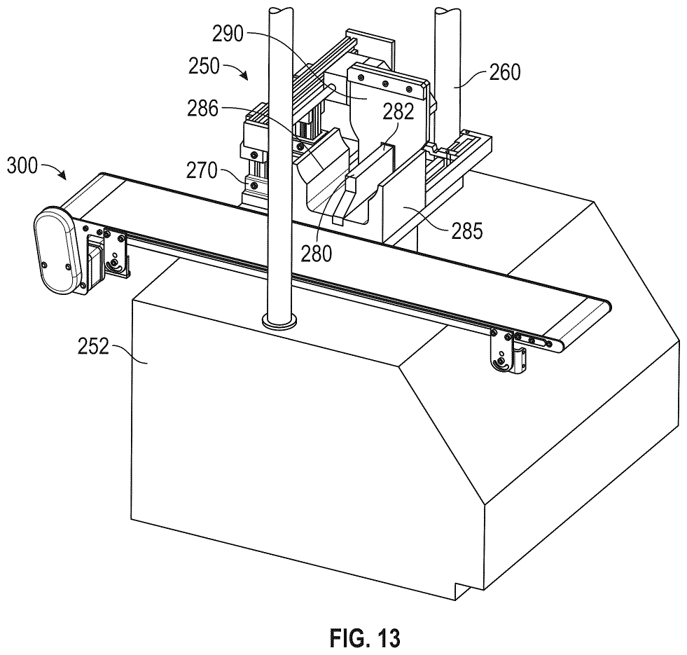

In this exemplary embodiment, holder assembly 270 includes a plurality of separate actuators for independently moving the cracking mount 280 and ejector unit 290 along a common horizontally extending axis. Particularly, holder assembly 270 is configured to move the cracking mount 280 between a retracted position (shown in FIGS. 10-12) and an extended position (shown in FIG. 13). The retracted position of cracking mount 280 corresponds to a first configuration of the tensile testing unit 250 whereby the tensile testing unit 250 is configured to perform an IDEAL-RT test on a material sample. Conversely, the extended position of cracking mount 280 corresponds to a second configuration of the tensile testing unit 250 whereby the tensile testing unit 250 is configured to perform an IDT strength or an IDEAL-CT test on a material sample. Additionally, the holder assembly 270 is configured to move the ejector unit 290 (comprising a plate, one or more actuators, and a conveyor belt in this exemplary embodiment) between a retracted position (shown in FIGS. 10, 11, and 13) and an extended position to thereby eject spent material samples from a receptacle of the tensile testing unit 250 defined by the cracking mount 280 or rutting mount 285 and onto the conveyor 300 which extends over a portion of the platform 252 in this exemplary embodiment.

In this exemplary embodiment, the cracking mount 280 is slidably positioned in a groove or track formed in the rutting mount 285 and the cracking mount 280 defines a planar contact surface 282 for contacting a material sample during an IDT strength test or an IDEAL-CT test performed by the tensile testing unit 250. Particularly, a material sample may be compressed between the contact surface 282 of cracking mount 280 and the stop 265 during the performance of an IDT strength test or an IDEAL-CT test. Additionally, in this exemplary embodiment, rutting mount 285 is stationary with respect to the holder assembly 270 and defines a pair of curved contact surfaces 287 against which a material sample is pressed during the performance of an IDEAL-RT test. Particularly, a material sample may be compressed between the pair of contact surfaces 287 of rutting mount 280 and the stop 265 of unit 250 during the performance of an IDEAL-RT test.

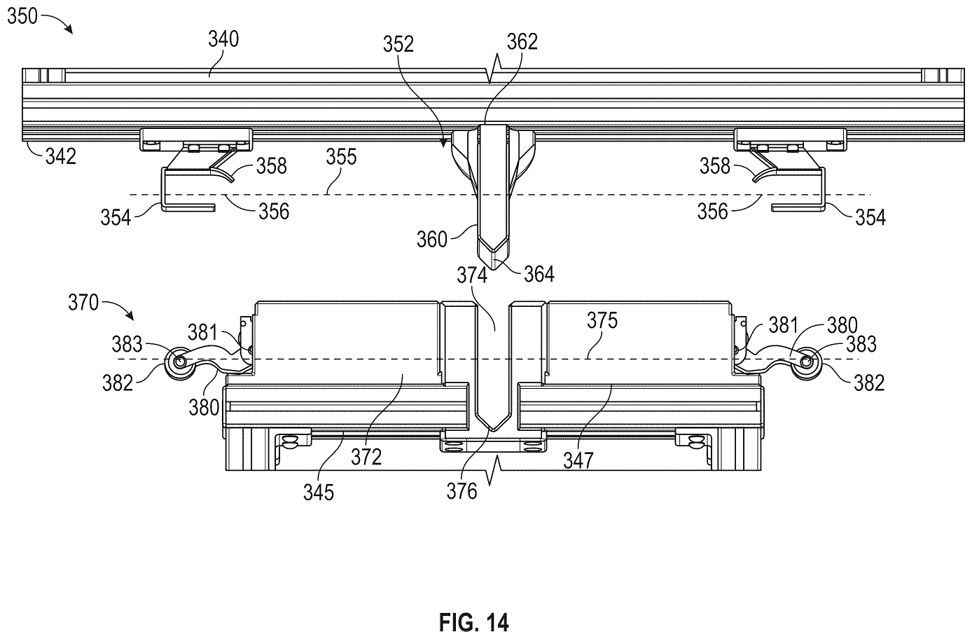

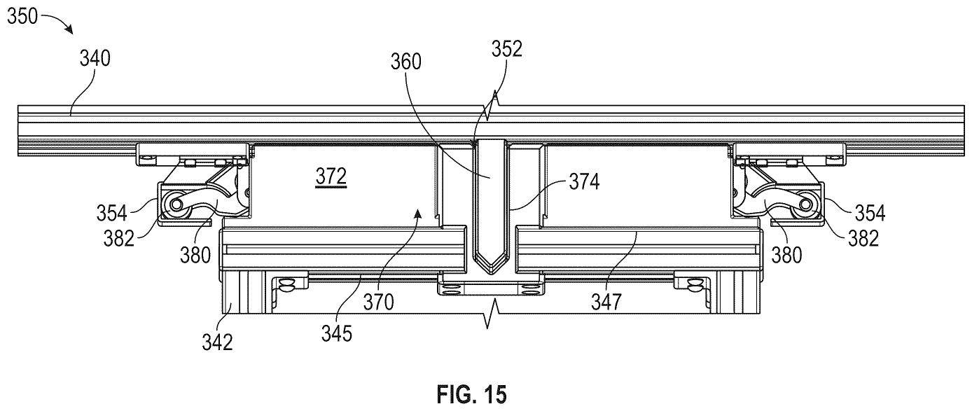

Referring now to FIGS. 14 and 15, an embodiment of a docking system 350 of a material testing system (e.g., material testing system 10 shown in FIG. 1) is shown. The successful implementation of automated material testing systems described herein (e.g., material testing system 10 shown in FIG. 1) is contingent on the robotic arm (e.g., robotic arm 15 shown in FIG. 1) being able to precisely locate the position of various units of the material testing system and material samples in physical space As but one example, the robotic arm must accurately locate the sample loading position (e.g., sample loading position 71 shown in FIG. 2) of a sample loading unit of the material testing system in order for the robotic arm to successfully grasp a selected material sample and load the material sample into the subsequent unit of the material testing system.

In order to address this challenge, automated robotic systems employing robotic arms often require precisely and laboriously determining the waypoints of salient features of the system (e.g., features to which the robotic arm interacts during operation) to properly calibrate the robotic arm such that these locations may be programmed into a controller of the system. However, this process is typically slow and laborious, thereby increasing the time required for assembling the given automated robotic system along with the costs for implementing the automated robotic system. In addition, some automated robotic systems are designed to be portable such that they may be operated at different locations (e.g., as part of different jobs or applications). With conventional automated robotic systems, this laborious calibration process may need to be performed each time the automated robotic system is assembled, thereby increasing the inconvenience and costs associated with operating the automated robotic system.

The docking system 350 shown in FIGS. 14-16 is intended to eliminate the need to repeatedly re-calibrate robotic waypoints for the automated robotic systems, including automated material testing systems, and particularly those systems which are intended to be portable and reassembled multiple times during their operational life. Instead, docking system 350 may be employed to precisely position in physical space one or more units of an automated robotic system in response to docking or coupling the different units of the system with a robotic arm of the system. In other words, docking system 350 requires the different units of a given automated robotic system to be properly positioned in calibrated, predefined positions in physical space relative to the robotic arm in order for these units to successfully work together.

In this exemplary embodiment, docking system 350 generally includes a first docking connector 352 and a corresponding second docking connector 370 configured to releasably couple with the first docking connector 352. The first docking connector 352 is coupled to a first platform 340 while the second docking connector 370 is coupled to a second platform 345. In some embodiments, the second platform 345 comprises an automated material testing unit. The first platform 340 may comprise the platform of a robotic arm or alternatively another unit of an automated robotic testing system while second platform 345 may comprise a platform of a unit of the automated robotic testing system or alternatively a platform of the robotic arm of the system. At least one of the platforms 340 and 345 are mobile to permit the second platform 345 to move relative to the first platform 340. As an example, the first platform 340 may comprise a platform of the robotic arm 15 shown in FIG. 1 while the second platform 345 may comprise a mobile platform or dolly of the void testing unit 30 shown in FIG. 1 (or another unit of material testing system 10) where docking system 350 permits the second platform 345 to dock with the first platform 340 whereby the second platform 345 is positioned in a predefined position in physical space relative to the first platform 345.

The first docking connector 352 is coupled or attached to a mounting surface 342 of first platform 340 located along an exterior of the platform 340 while the second docking connector 370 is similarly coupled or attached to a mounting surface 347 of the second platform 345 located along an exterior thereof. The mounting surface 347 of second platform 345 opposes the mounting surface 342 of first platform 340 as the docking connectors 352 and 370 are coupled together to dock the first platform 340 with the second platform 345.

In this exemplary embodiment, first docking connector 352 comprises a pair of receptacles 354 and a central tongue 360 located between the pair of receptacles 354 along a first connector axis 355 of the first docking connector 352 (and which extends parallel to the mounting surface 342 of first platform 340 in this exemplary embodiment. Each receptacle 354 is generally U-shaped and includes an opening 356 aligned with the first connector axis 355 such that axis 355 extends into and through the openings 356 of receptacles 354. In addition, in this exemplary embodiment, each receptacle 354 includes a curved insertion shoulder 358 located adjacent the opening 356 to assist in facilitating (e.g., to make more convenient or easier) the process of coupling the first docking connector 352 with the second docking connector 370 as will be further described herein.

The tongue 360 is located along first connector axis 355 between receptacles 354. Particularly, tongue 360 extends from a first or fixed end 362 coupled to the mounting surface 342 of first platform 340 to a longitudinally opposed second or free end 364. Additionally, tongue 360 projects orthogonally from mounting surface 342 such that a central or longitudinal axis of tongue 360 is oriented orthogonal the first connector axis 355. Further, in this exemplary embodiment, the free end 364 of tongue 360 has a cross-section that increases in width and area moving from a tip of the free end 364 towards the fixed end 362 of tongue. In this exemplary embodiment, free end 364 is defined by a pair of inclined surfaces providing free end 364 with a wedge shape; however, in other embodiments, the configuration of free end 364 may vary. By increasing in the cross-sectional area and/or width moving from the tip of free end 364 towards the fixed end 362, tongue 360 may improve the ease or convenience in coupling the first docking connector 352 with the second docking connector 370 as will be discussed further herein.

In this exemplary embodiment, the second docking connector 370 of docking system 350 generally includes a base 372 and a pair of locking members or locks 382 pivotably coupled to the base 372. Particularly, in this exemplary embodiment, base 372 extends longitudinally along a second connector axis 375 of second docking connector 370 that extends parallel to the mounting surface 347. In addition, a groove 374 is formed in base 372 which extends orthogonal to the second connector axis 372 and terminates at a terminal end 376. In this exemplary embodiment, the terminal end 376 of groove 374 has a cross-section that increases in width and area moving from a tip of the terminal end 376 towards the opposing longitudinal end of groove 374. In this exemplary embodiment, terminal end 376 is defined by a pair of inclined surfaces providing terminal end 376 with a wedge shape; however, in other embodiments, the configuration of terminal end 376 may vary. The wedge-shaped terminal end 376 of groove 374 matches the wedge-shaped free end 364 of the tongue 360 of first docking connector 352 whereby tongue 360 may be matingly received in groove 374 when docking connectors 352 and 370 are coupled together.

The locks 382 of second docking connector 370 are receivable in the receptacles 354 of first docking connector 352 to secure or lock the second docking connector 370 with the first docking connector 352. Particularly, the pair of locks 382 are pivotably coupled to the base 372 of second docking connector 370 via a pair of corresponding pivot arms 380 each extending between a first or pivot end pivotably coupled to the base 372 at a first joint 381, and a second end opposite the pivot end 381 and coupled to one of the locks 382 at a second joint 383. In this exemplary embodiment, joints 381 and 383 each comprise pivot or rotational joints such that arms 380 are pivotable relative to the base 372 at the first joints 381 while locks 382 are pivotable relative arms 380 about joints 383. In this exemplary embodiments, locks 382 comprise rolling elements or rollers rotatably mounted to arms 380 at second joints 383; however, it may be understood that the configuration of locks 382 may vary in other embodiments (e.g., locks 382 may not comprise rollers and may not be pivotable/rotatable relative to arms 380).

Locks 382 may be rotated about first joints 381 between an unlocked position (shown in FIG. 14) and a locked position (shown in FIG. 15) that is spaced (e.g., circumferentially spaced about first joints 381) from the unlocked position. With locks 382 positioned in their unlocked positions, second docking connector 370 may be coupled with first docking connector 352 whereby the locks 382 are transitioned automatically from their unlocked positions to their locked positions. Particularly, initially second docking connector 370 may be aligned with first docking connector 352 as shown in FIG. 14 with tongue 360 of first docking connector 352 aligned with groove 374 of second docking connector 370 along a shared or common longitudinal axis. In this position, tongue 360 may be inserted longitudinally into groove 374 with the wedge-shaped free end 364 of tongue 360 assisting in precisely aligning tongue 360 with groove 374.

As tongue 360 of first docking connector 352 is inserted into the groove 374 of second docking connector 370, the locks 382 (disposed in their unlocked positions), contact the curved insertion shoulders 358 of receptacles 354. Given that insertion shoulders 358 extend at a non-zero angle (e.g., an acute angle) relative to connector axes 355 and 375, contact between insertion shoulders 358 and locks 382 applies a moment or torque against arms 380 urging locks 382 towards their locked positions. In this manner, locks 382 are rotated in response to contact with insertion shoulders 358 from their unlocked positions to their locked positions as tongue 360 is inserted into groove 374, thereby coupling or securing the docking connectors 352 and 370 together. In some embodiments, locks 382 are lockable (e.g., via a handle or other mechanism) into their locked positions such that second docking connector 370 may not be inadvertently released from first docking connector 352.

Comprising rollers, locks 382 may roll along the walls of receptacles 354 as locks 382 are transitioned into their locked positions. This rolling contact between locks 382 and receptacles 354 minimizes or eliminates the need for “play” between locks 382 and receptacles 354 such that second docking connector 370 may be precisely located in a predefined position relative to first docking connector 352 when docking connectors 352 and 370 are coupled together. This lack of play and resulting precision in the location of second docking connector 370 relative to first docking connector 352 ensures that a robotic arm supported by either of platforms 340 and 345 may successfully interact (e.g., grasp) with features supported by the other of platforms 340 and 345 without the need for recalibrating the robotic arm.

In some instances, at least some of the units of an automated robotic system may not be directly connected to the platform supporting the robotic arm of the robotic system. Instead, at least some unit may be connected to the robotic arm through one or more other units coupled between the robotic arm and the given unit. In such applications, the unit connected to the robotic arm through the intervening unit of the robotic system must be precisely positioned relative to the intervening unit to ensure the robotic arm may successfully interact with the respective unit (e.g., accurately locate in physical space desired features of the respective unit). The connections formed directly between the platform of the robotic arm of the robotic system and surrounding units of the robotic system (e.g., the connection formed between docking connectors 352 and 370 shown in FIGS. 14 and 15) may be referred to herein as primary connections herein while connections formed between the units themselves and not directly with the platform of the robotic arm may be referred to herein as secondary connections.

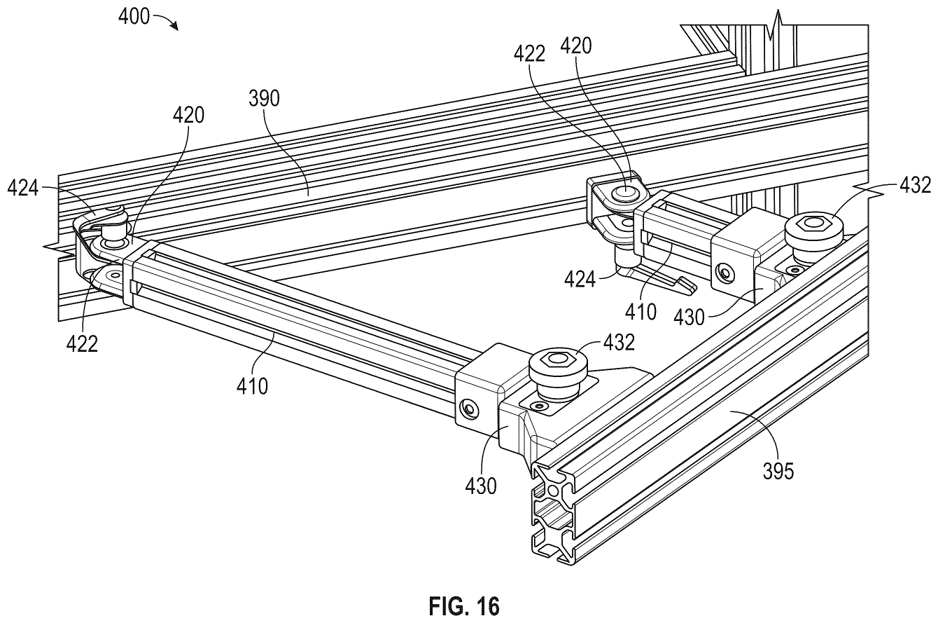

Referring now to FIG. 16, a secondary connection 400 of a docking system (e.g., docking system 350 shown in FIGS. 14 and 15) is shown. As described above, secondary connection 400 may be used to directly connect a first unit of an automated robotic system with a second unit of the system. Particularly, secondary connection 400 extends between a first platform (e.g., a mobile platform or dolly) 390 and a second platform (e.g., another mobile platform or dolly) 395 that is spaced and separate from the first platform 390.

In this exemplary embodiment, secondary connection 400 comprises one or more connector arms 410 extending between and coupling together the first platform 390 with the second platform 395. Each of the connector arms 410 is pivotably connected at a first end thereof to a mounting surface of the first platform 390 via a pivot mount 420 of the secondary connection 400. Each pivot mount 420 comprises a pivot joint 422 permitting the connector arms 410 to pivot about pivot axes which extend orthogonally to longitudinal or central axes of the connector arms 410. In addition, pivot mounts 420 comprise pivot locks 424 configured to selectably lock the pivot joints 422 of pivot mounts 420. In this manner, connector arms 410 may be locked into desired and predefined angular orientations relative to the first platform 390.

In this exemplary embodiment, secondary connection 400 also includes one or more telescoping mounts 430 coupled or attached to a mounting surface of the second platform 395. The longitudinal lengths of connector arms 410 are adjustable telescopically via the telescoping mounts 430 which are coupled to the second ends (longitudinally opposite the first ends to which pivot mounts 420 are coupled) of connector arms 410. In addition, in this exemplary embodiment, telescoping mounts 430 comprise telescoping locks 432 for selectably locking (e.g., via turning the telescoping locks 432) the longitudinal lengths of connectors arms 410. Thus, pivot locks 424 may be locked to lock the angular positions of connector arms 410 relative to first platform 390 while telescoping locks 432 may be locked to lock the longitudinal lengths of connector arms 410 thereby restricting relative movement between platforms 390 and 395. In this manner, platforms 390 and 395 may be positioned in desired, predefined positions in physical space relative to one another with secondary connection 400 in an unlocked state (e.g., with each of locks 424 and 432 unlocked). Once in their predefined positions, secondary connection 400 may be transitioned to a locked state (e.g., with each of locks 424 and 432 locked) to lock platforms 390 and 395 into their predefined relative positions.

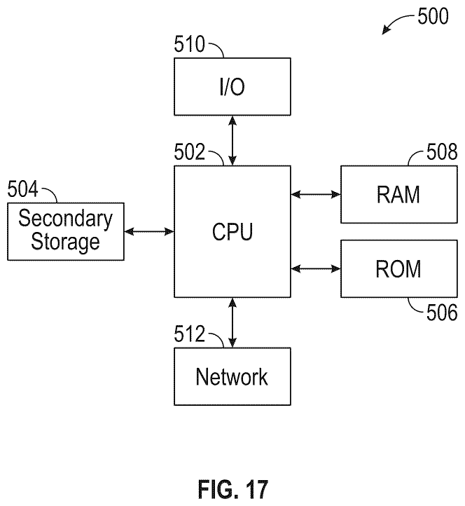

Referring now to FIG. 17, an embodiment of a computer system 500 is shown suitable for implementing one or more components disclosed herein. As an example, computer system 500 may be used to execute various embodiments of material testing systems (e.g., material testing system 10 shown in FIG. 1) disclosed herein. As an example, the computer system 500 may comprise an embodiment of the system controller shown in FIG. 1.

The computer system 500 of FIG. 17 generally includes a processor 502 (which may be referred to as a central processor unit or CPU) that is in communication with memory devices including secondary storage 504, read only memory (ROM) 506, random access memory (RAM) 508, input/output (I/O) devices 510, and network connectivity devices 512. The processor 502 may be implemented as one or more CPU chips. It is understood that by programming and/or loading executable instructions onto the computer system 500, at least one of the CPU 502, the RAM 508, and the ROM 506 are changed, transforming the computer system 500 in part into a particular machine or apparatus having the novel functionality taught by the present disclosure.

Additionally, after the system 500 is turned on or booted, the CPU 502 may execute a computer program or application. For example, the CPU 502 may execute software or firmware stored in the ROM 506 or stored in the RAM 508. In some cases, on boot and/or when the application is initiated, the CPU 502 may copy the application or portions of the application from the secondary storage 504 to the RAM 508 or to memory space within the CPU 502 itself, and the CPU 502 may then execute instructions that the application is comprised of. In some cases, the CPU 502 may copy the application or portions of the application from memory accessed via the network connectivity devices 512 or via the I/O devices 510 to the RAM 508 or to memory space within the CPU 502, and the CPU 502 may then execute instructions that the application is comprised of. During execution, an application may load instructions into the CPU 502, for example load some of the instructions of the application into a cache of the CPU 502. In some contexts, an application that is executed may be said to configure the CPU 502 to do something, e.g., to configure the CPU 502 to perform the function or functions promoted by the subject application. When the CPU 502 is configured in this way by the application, the CPU 502 becomes a specific purpose computer or a specific purpose machine.

Secondary storage 504 may be used to store programs which are loaded into RAM 508 when such programs are selected for execution. The ROM 506 is used to store instructions and perhaps data which are read during program execution. ROM 506 is a non-volatile memory device which typically has a small memory capacity relative to the larger memory capacity of secondary storage 504. The secondary storage 504, the RAM 508, and/or the ROM 506 may be referred to in some contexts as computer readable storage media and/or non-transitory computer readable media. I/O devices 510 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 512 may take the form of modems, modem banks, Ethernet cards, universal serial bus (USB) interface cards, wireless local area network (WLAN) cards, radio transceiver cards, and/or other well-known network devices. The network connectivity devices 512 may provide wired communication links and/or wireless communication links. These network connectivity devices 512 may enable the processor 502 to communicate with the Internet or one or more intranets. With such a network connection, it is contemplated that the processor 502 might receive information from the network, or might output information to the network. Such information, which may include data or instructions to be executed using processor 502 for example, may be received from and outputted to the network, for example, in the form of a computer data baseband signal or signal embodied in a carrier wave.

The processor 502 executes instructions, codes, computer programs, scripts which it accesses from hard disk, floppy disk, optical disk, flash drive, ROM 506, RAM 508, or the network connectivity devices 512. While only one processor 502 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 504, for example, hard drives, floppy disks, optical disks, and/or other device, the ROM 506, and/or the RAM 508 may be referred to in some contexts as non-transitory instructions and/or non-transitory information.

In an embodiment, the computer system 500 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, the functionality disclosed above may be provided by executing the application and/or applications in a cloud computing environment. Cloud computing may comprise providing computing services via a network connection using dynamically scalable computing resources.

While embodiments of the disclosure have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

1. An automated material testing system, the system comprising:
a robotic arm comprising a base and a gripper pivotably coupled to the base, wherein the gripper has a defined field of motion;
a void testing unit positioned at least partially within the field of motion of the gripper of the robotic arm, the void testing unit comprising a scale for weighing a material sample in both a dry state and a saturated surface dry (SSD) state;
a material properties testing unit positioned at least partially within the field of motion of the gripper of the robotic arm, the materials property testing unit comprising sample mount for supporting a material sample during the performance of a material properties test on the material sample; and
a controller comprising a processor and storage for storing instructions executable by processor to operate the robotic arm, the void testing unit, and the material properties testing unit.

2. The system of claim 1, wherein the material properties testing unit comprises a tensile testing unit comprising at least one of a cracking mount and a rutting mount for performing at least one of an indirect tensile (IDT) strength test, an indirect tensile asphalt cracking test (IDEAL-CT), and an ideal rutting test (IDEAL-RT) on the material sample.

3. The system of claim 2, wherein the tensile testing unit comprises:
a platform;
a holder assembly supported on the platform and defining a receptacle configured to receive the material sample, the holder assembly comprising a cracking mount, a rutting mount, and an ejector unit; and
a tensile load applicator coupled between the holder assembly and the platform and configured to move the holder assembly relative to the platform;
wherein the cracking mount is moveable by an actuator of the holder assembly relative to the rutting mount.

4. The system of claim 3, wherein the cracking mount comprises an extended position corresponding to a first configuration of the tensile testing unit whereby the tensile testing unit is configured to perform either the IDT strength test or the IDEAL-CT, and a retracted position corresponding to a second configuration of the tensile testing unit whereby the tensile testing unit is configured to perform the IDEAL-RT.

5. The system of claim 1, further comprising a conditioning unit positioned at least partially within the field of motion of the gripper of the robotic arm, the conditioning unit configured to adjust the temperature of the material sample, without manual intervention, to meet a desired temperature.

6. The system of claim 1, further comprising a sample drying unit positioned at least partially within the field of motion of the gripper of the robotic arm, the sample drying unit configured to dry the exterior of the material sample without manual intervention.

7. The system of claim 6, wherein the sample drying unit comprises a towel belt and a drive unit configured to transport the towel belt along a closed loop path.

8. The system of claim 1, further comprising a radio-frequency identification (RFID) tracking system comprising a RFID reader configured to read one or more RFID tags attached to one or more of the material samples to identify the one or more material samples.

9. An automated testing system, the system comprising:
a robotic arm comprising a base and a gripper pivotably coupled to the base, wherein the gripper has a defined field of motion;
an automated loading system comprising a carousel within the field of motion of the robotic arm and having a plurality of receptacles for receiving one or more material samples, wherein the robotic arm is configured to retrieve the one or more material samples from the plurality of receptacles of the carousel;
a material properties testing unit positioned at least partially within the field of motion of the gripper of the robotic arm, the materials property testing unit comprising sample mount for supporting a material sample during the performance of a material properties test on the material sample; and
a controller comprising a processor and storage for storing instructions executable by processor to operate the robotic arm, the material properties testing unit, and the automated loading system.

10. The system of claim 9, wherein the loading system comprises a carousel upon which the plurality of receptacles are arranged, the carousel being rotatable about a rotational axis.

11. The system of claim 10, wherein the loading system comprises a carousel motor in signal communication with the controller, and wherein the controller is configured to operate the carousel motor to rotate the carousel about the rotational axis and position a selected receptacle in a predefined loading position.

12. The system of claim 10, wherein the carousel has a plurality of separate tiers spaced along the rotational axis, wherein at least some of the plurality of receptacles are arranged along each of the plurality of separate tiers.

13. The system of claim 9, further comprising a void testing unit positioned at least partially within the field of motion of the gripper of the robotic arm, the void testing unit comprising a scale for weighing a material sample in both a dry state and a saturated surface dry (SSD) state.

14. The system of claim 9, wherein the material properties testing unit comprises a tensile testing unit comprising at least one of a cracking mount and a rutting mount for performing at least one of an indirect tensile (IDT) strength test, an indirect tensile asphalt cracking test (IDEAL-CT), and an ideal rutting test (IDEAL-RT) on the material sample.

15. The system of claim 9, further comprising a radio-frequency identification (RFID) tracking system comprising a RFID reader configured to read one or more RFID tags attached to one or more of the material samples to identify the one or more material samples.

16. An automated material testing system, the system comprising:
a robotic arm comprising a base and a gripper pivotably coupled to the base, wherein the gripper has a defined field of motion;
a radio-frequency identification (RFID) tracking system comprising a RFID reader configured to read one or more RFID tags attached to one or more corresponding material samples to identify the one or more material samples;
a material properties testing unit positioned at least partially within the field of motion of the gripper of the robotic arm, the materials property testing unit comprising sample mount for supporting a material sample during the performance of a material properties test on the material sample; and
a controller comprising a processor and storage for storing instructions executable by processor to operate the robotic arm, the material properties testing unit, and the RFID tracking system.

17. The system of claim 16, wherein the controller is configured to correlate a test result of the material properties testing unit with a specific RFID tag of the one or more RFID tags to identify a specific material sample of the one or more material samples corresponding to the test result.

18. The system of claim 16, wherein the RFID reader is mounted at a predefined position for reading the one or more RFID tags.

19. The system of claim 16, wherein the one or more RFID tags stores identifying information of the one or more corresponding material samples.

20. The system of claim 16, wherein the material properties testing unit comprises a tensile testing unit comprising at least one of a cracking mount and a rutting mount for performing at least one of an indirect tensile (IDT) strength test, an indirect tensile asphalt cracking test (IDEAL-CT), and an ideal rutting test (IDEAL-RT) on the material sample.

21. The system of claim 16, further comprising a void testing unit positioned at least partially within the field of motion of the gripper of the robotic arm, the void testing unit comprising a scale for weighing a material sample in both a dry state and a saturated surface dry (SSD) state.
